(19) United States
(12) Patent Application Publication
(10) Pub. No.: US 2023/0293439 A1
(43) Pub. Date: Sep. 21, 2023

(54) SPRAY DRYING OF SUPERSATURATED SOLUTIONS OF API WITH ACETIC ACID

(71) Applicant: BEND RESEARCH, INC., Bend, OR (US)

(72) Inventors: Molly Adam, Bend, OR (US); John Baumann, Bend, OR (US); Nathan Bennette, Bend, OR (US); Warren Miller, Bend, OR (US); Michael Morgen, Bend, OR (US); Amanda Pluntze, Bend, OR (US); Daniel Regan, Bend, OR (US); David Vodak, Bend, OR (US)

(73) Assignee: Lonza Bend Inc., Bend, OR (US)

(21) Appl. No.: 18/006,549

(22) PCT Filed: Jul. 22, 2021
(86) PCT No.: PCT/EP2021/070577

§ 371 Date: Jan. 23, 2023

Related U.S. Application Data
(60) Provisional application No. 63/056,094, filed on Jul. 24, 2020.

(30) Foreign Application Priority Data
Aug. 24, 2020 (EP) 20192298.6
Aug. 25, 2020 (EP) 20192561.7
Sep. 22, 2020 (EP) 20197562.0
Oct. 2, 2020 (EP) 20199830.9
Apr. 7, 2021 (EP) 21167282.9
Jun. 10, 2021 (EP) 21178673.6

(51) Int. Cl. A61K 9/16; A61K 45/06

(52) U.S. Cl. CPC A61K 9/1682; A61K 45/06; A61K 9/1635; A61K 9/1652

(57) ABSTRACT

The invention discloses a method for preparation of spray dried solid dispersions (SDD) comprising an active pharmaceutical ingredient (API) and a dispersion polymer (DISPPOL), wherein the spray drying is done with a supersaturated solution of API in a solvent mixture comprising two solvents, one is acetic acid, this supersaturated solution further comprising DISPPOL.

The invention discloses a method for preparation of spray dried solid dispersions (SDD) comprising an active pharmaceutical ingredient (API) and a dispersion polymer (DISPPOL), wherein the spray drying is done with a supersaturated solution of API in a solvent mixture comprising two solvents, one is acetic acid, this supersaturated solution further comprising DISPPOL.

### BACKGROUND OF THE INVENTION

Spray dried solid dispersions (SDD) comprising an active pharmaceutical ingredient (API) and a dispersion polymer (DISPPOL) are typically produced by dissolving the dispersion polymer and the API in a volatile solvent, such as methanol or acetone, or in a mixture of solvents, followed by spray drying. In cases where the API has limited solubility, e.g. <1 wt %, in the spray drying solvent, an API suspension can be heated to a temperature either below or above the solvent's ambient pressure boiling point, this is known as “hot spray drying process”, resulting in a higher dissolved concentration of API. In some cases, even the higher temperatures do not give adequate API concentrations that are economical as a spray drying process, or cause other problems such as chemical degradation of the API, or the potential for incomplete API dissolution in the heat exchanger. Alternate, non-preferred volatile solvents can provide increased solubility of the API, but these solvents have other disadvantages that make them less desirable, e.g. high cost, toxicity, poor equipment compatibility, poor commercial availability, high disposal costs, challenges removing to sufficiently low levels.

Spray drying of suspensions is usually avoided since suspensions can lead to clogging of the nozzle of the spray dryer. Furthermore, when the intent of spray drying is the provision of an amorphous solid dispersion (ASD) of an API in a dispersion polymer, then this target is best achieved when both the API and the dispersion polymer are dissolved in the spray drying solvent so that both of them are not present in sold form in the spray drying mixture; thereby the desired intimate, homogenous and amorphous mixture of the ASD with the dispersion polymer is best obtained.

WO 2019/220282 A1 discloses in Example 1 spray drying of a solution of erlotinib and a dispersion polymer (PMMAMA or hydroxypropyl methylcellulose acetate succinate H grade) in methanol to provide a spray dried dispersion.

US 2020/261449 A1 discloses amorphous solid dispersions of nilotinib fumarate or nilotinib tartrate. In example 13 “the required quantities of nilotinib fumarate, fumaric acid and HPMC-AS were dissolved in methanol solvent to prepare a solution containing 3% solid content. Prepared solution was sprayed on a spray dryer”. The solution contained according tot TABLE 19 60.77 mg nilotinib fumarate, 182.3 mg HPMC-AS MF, 50 mg fumaric acid and 9′000 mg methanol, giving a total weight of the 9′293.07 mg. The term “3% solid content” refers to the content of solids dissolved in the solution, which is [60.77 mg nilotinib fumarate plus 182.3 mg HPMC-AS MF equals 243.078 mg] divided by 9′293.07 mg equals 3%.

So a conventional solution was used for spray drying, wherein all solids were dissolved. US 2020/261449 A1 does not disclose a supersaturated feed solution for spray drying.

US 2009/247468 A1 discloses in [0007] an effect of a non-volatile or high-boiling solvent as a component in the feed solution for spray drying on the properties of the resulting spray dried particles, such as increase of size, density or flowability. Claim 2 specifies that the mixture used as feed for spray drying is a solution or a suspension. A supersaturated solution of a compound is thermodynamically metastable. Nucleation and ensuing precipitation are kinetically blocked. But a suspension of a compound in a solvent cannot be or contain simultaneously a supersaturated solution, since any solid particles of the compound in the suspension would cause crystallization of any dissolved amount of the compound in the solution which exceed the maximum solubility of the compound in the solvent, that is any solid particles of the compound which are present in a supersaturated solution would act as nucleation factor, overcoming the kinetic barrier, and would cause nucleation and precipitation of any amount of compound dissolved above the maximum solubility, thereby leading to a thermodynamically stable state, the suspension. Therefore a suspension of the compound in a solvent can at most be a mixture of the solid compound, which is suspended, together with the solvent containing dissolved compound at most as much as a saturated solution can contain the compound.

in connection with [0164] discusses possible dissolution characteristics of compounds in gastric fluid where a state of supersaturation may be transiently passed during the dissolution of the compound in the stomach. But this clearly refers to a dissolution behavior of the ASD in the stomach, not to the spray drying.

For these reasons US 2009/247468 A1 does not disclose supersaturated feed solutions for spray drying.

WO 2019/220282 A1 discloses oral pharmaceutical compositions comprising a solid dosage form (SDF). Supersaturation is disclosed in the abstract, on page 6 line 29 or on page 12 line 39 and other places only in connection with the SDF itself and with a rapid disintegration of the SDF in the use environment, that is within a patient. Example 1 discloses spray solutions with a solids loading of 3%, but again this term “solids loading” refers to the amount of solids dissolved in the solution.

Therefore WO 2019/220282 A1 does not disclose supersaturated feed solutions for spray drying.

Paudel et al, INTERNATIONAL JOURNAL OF PHARMACEUTICS, ELSEVIER, NL, 2021, 453, 253-284, discloses supersaturation of the API in carrier matrix, that is a supersaturation in the solid mixture which is obtained by the spray drying, the solid dispersion, see the third part of point 1, Introduction: “. . . The very fast solvent evaporation during spray drying leads to rapid viscosity increase and permits kinetic trapping of the API in the carrier matrix. Often a (supersaturated) molecular dispersion is the result of this process . . . ”. The same result, that is possible supersaturation of the drug in carrier matrix, is meant under point 5.1.1 “Feed composition” in the first second sentence: “. . . The solubility difference among drug, carrier and other additives in a feed solution (solvent) leads to a different degree of saturation/supersaturation of these components . . . ”; the term “leads” clearly indicates that any possible supersaturation may occur in the solid dispersion to which the spray drying leads to.

Similar to WO 2019/220282 A1, supersaturation is mentioned that it may also occur and be maintained during the in vitro dissolution in the gastro intestine, see inter alia second have of the first paragraph of point 4.1: “. . . the supersaturation generated during in vitro dissolution and after oral administration in the gastrointestinal milieu . . . ”.

Therefore also Paudel et al does not disclose supersaturation in the feed solution of the spray drying.

Super-solvents such as acetic acid can dissolve APIs to high concentration. However, it can be disadvantageous to prepare such SDD from pure acetic acid, due to the high viscosity of a solution containing also a high concentration of dissolved dispersion polymer, poor solubility of desired excipients in acetic acid, poor atomization/drying characteristics, or safety aspects of the acetic acid.

There was a need for a method for preparing spray dried solid dispersion of API and dispersion polymers, which allows for dissolving the APIs in easily processable spray drying solvents at modest temperature, i.e. a temperature below the ambient pressure boiling point, at sufficiently high concentrations to enable economical throughput of SDDs.

A Solvent-Shift route was found which involves dissolving API to a high concentration in acetic acid, thereby providing a solution of the API in acetic acid having a relatively high viscosity, then diluting this API solution with a preferred spray drying solvent having a relatively low viscosity, e.g. methanol, ethanol or acetone, and containing the desired dispersion polymer, at ratios that result in a metastable supersaturated solution of API in a solvent mixture containing the dispersion polymer and having a rather low viscosity and that can be efficiently spray dried. Metastable supersaturated solution of API in the solvent mixture means that the API is present in the solvent mixture in dissolved state, no solid API is present. In a supersaturated solution the API is present at a concentration above the thermodynamical equilibrium concentration.

Such a supersaturated solution cannot be prepared by simply adding API to the solvent mixture; it must be generated by mixing of two solvents containing dissolved API and dispersion polymer, respectively. An advantage is that by the dilution of the solution of the API in acetic acid with the preferred spray drying solvent, the viscosity of the resulting metastable supersaturated solution can be chosen to be comparably as low as the viscosity of the pure preferred spray drying solvent. Another advantage of the Solvent-Shift route is that it allows higher concentration of dissolved API in the spray drying solution relative to thermodynamic maximum solubility of the API in the spray drying solution, giving higher spray drying efficiency and higher throughput for the manufacturing of SDDs of APIs, especially for such APIs with a rather low solubility in typical spray drying solvents. The higher API and dispersion polymer concentrations in the spray drying solution may also allow for enhanced properties of the spray dried particles, e.g. larger particles giving advantages for dosage form manufacture or product recovery.

### ABBREVIATIONS AND DEFINITIONS USED IN THIS SPECIFICATION

AA active agent
API active pharmaceutical ingredient
DISPPOL dispersion polymer
glacial acetic acid water-free (anhydrous) acetic acid, acetic acid 100%
HPMCAS Hydroxypropyl Methylcellulose Acetate Succinate, Hypromellose Acetate Succinate, CAS 71138-97-1
MIXSOL2DISPPOL a mixture of DISPPOL with a second solvent SOL2
pKa the pKa of a basic site of an organic Bronstedt base is the pH at which half of these basic sites are protonated. At a pH which is lower than this basic pKa more than half of these basic sites are protonated, that is ionized. This pKa of a basic site is also called basic pKa.
In contrast thereof the pKa of an acidic site of an organic Bronstedt acid is the pH at which half of these acidic sites are deprotonated, that is ionized. At a pH which is higher than this acidic pKa more than half of these acidic sites are deprotonated. This pKa of an acidic site is also called acidic pKa.
   pKa values are available in the internet, they may also be calculated, for example by ADMET predictor® software, Simulations Plus, Inc. (Nasdaq: SLP) or measured in the lab.
PPO polypropylenoxide
room temperature about 20° C.

PXRD Powder X-Ray Diffraction
SDD Spray dried solid dispersions
SOL1 first solvent
SOL2 second solvent
SOLUTION1 a solution of AA in a first solvent SOL1, optionally further comprising DISPPOL
SUPSATSOL supersaturated solution of an active agent AA in a solvent mixture SOLMIX further comprising DISPPOL
SOLMIX the solvent mixture of SOL1 and SOL2 which is obtained when SOLUTION1 and MIXSOL2DISPPOL or SOL2 are mixed
Vitamin E TPGS Tocofersolan (INN), Vitamin E D-α-tocopheryl polyethylene glycol succinate
wt % any wt % value herein is based on the weight of the solution or mixture, if not explicitly stated otherwise

### SUMMARY OF THE INVENTION

Subject of the invention is a method SPRAYDRY for preparing a spray dried solid dispersion SDD comprising an active agent AA and a dispersion polymer DISPPOL;

the method SPRAYDRY comprising:
providing a solution SOLUTION1 of AA in a first solvent SOL1,
mixing SOLUTION1 with a second solvent SOL2 to provide a solution SUPSATSOL,
spray drying of SUPSATSOL in a spray dryer;
   wherein
   AA is a drug, medicament, pharmaceutical, therapeutic agent, nutraceutical or an active pharmaceutical ingredient;
   SUPSATSOL comprises a solvent mixture SOLMIX and AA, with SOLMIX being the mixture of SOL1 and SOL2;
   SUPSATSOL is a supersaturated solution of AA in SOLMIX;
   SUPSATSOL does not contain AA in solid form;
   DISPPOL is contained in SOLUTION1, in SOL2 or in both prior to the mixing of SOLUTION1 with SOL2,
   SOL1 comprises from 90 to 100 wt % of acetic acid, with the wt % being based in the weight of SOl1;
   AA is stable in SOL1, SOL2 and SOLMIX.

### DETAILED DESCRIPTION OF THE INVENTION

Supersaturation in the sense of the invention means a concentration of AA in SOLMIX which is above the concentration of a saturated solution of AA in SOLMIX at a given temperature, in particular at the temperature of SUPSATSOL when SUPSATSOL is fed into the spray dryer; so the concentration of AA in SOLMIX is above the respective thermodynamical equilibrium concentration of AA in SOLMIX. SUPSATSOL is a metastable supersaturated solution of AA in SOLMIX. Metastable in the sense of the invention means that AA does not precipitate from SUPSATSOL between the preparation of SUPSATSOL and its spray drying. So AA is present in SUPSATSOL in a completely dissolved state. SUPSATSOL does not contain AA in solid form.

SUPSATSOL has preferably only one liquid phase.

The amounts of AA, SOLMIX and DISPPOL may be chosen respectively. Supersaturation of AA in SUPSATSOL may also be expressed relative to the solubility of AA in SOLMIX; the concentration of AA in SUPSATSOL may be at least 1.1-fold, preferably at least 1.5-fold, more preferably at least 2-fold, even more preferably at least 5-fold, especially at least 10-fold, of the concentration of a saturated solution of AA in SOLMIX at a given temperature, in particular at the temperature of SUPSATSOL when SUPSATSOL is fed into the spray dryer.

Possible amounts of AA in SUPSATSOL may be from 0.5 wt % to 10 wt %, preferably from 1 wt % to 7.5 wt %, more preferably from 1 wt % to 5 wt %, with the wt % being based on the weight of SUPSATSOL.

When DISPPOL is comprised in SOL2 prior to the mixing of SOLUTION1 with SOL2 then this mixture of DISPPOL with SOL2 is called MIXSOL2DISPPOL herein.

Therefore,
in one embodiment, SUPSATSOL is prepared by mixing SOLUTION1, which is a solution of AA in SOL1, with MIXSOL2DISPPOL;
in another embodiment, SUPSATSOL is prepared by mixing SOLUTION1, which is a solution of AA in SOL1 comprising DISPPOL, with SOL2;
in yet another embodiment, SUPSATSOL is prepared by mixing SOLUTION1, which is a solution of AA in SOL1 comprising DISPPOL, with MIXSOL2DISPPOL;
preferably, SUPSATSOL is prepared by mixing SOLUTION1, which is a solution of AA in SOL1, with MIXSOL2DISPPOL.

The mixing of SOLUTION1 with MIXSOL2DISPPOL or with SOL2 to prepare SUPSATSOL may be done in any way that is known to the skilled person for the mixing of liquids, such as continuous mixing, for example by with an in-line mixer, such as a T shaped mixer, or by batch wise mixing, for example in a vessel.

In case of continuous mixing, the mixing and the spray drying of SUPSATSOL may be done continuously and consecutively, that is without any isolation or retainment of SUPSATSOL between the mixing and the spray drying. Thereby the time between the mixing and the spray drying of SUPSATSOL may be short, this time may be as short as a few milliseconds to seconds; this may be advantageous in case that the metastability of SUPSATSOL is only of short duration.

The mixing of SOLUTION1 with MIXSOL2DISPPOL or with SOL2 to prepare SUPSATSOL may be done
with SOLUTION1 having a temperature of from 4° C. to the boiling point of SOLUTION1 at ambient pressure, preferably from 4° C. to a temperature below the boiling point of SOLUTION1 at ambient pressure, and
with MIXSOL2DISPPOL or SOL2 having a temperature of from 4° C. to the boiling point of MIXSOL2DISPPOL at ambient pressure, preferably from 4° C. to a temperature below the boiling point of MIXSOL2DISPPOL at ambient pressure;

preferably
with SOLUTION1 having a temperature of from 4 to 60° C., preferably from room temperature to 60° C., and
with MIXSOL2DISPPOL or SOL2 having a temperature of from 4 to 60° C., preferably from room temperature to 60° C.

SOLMIX is the solvent mixture of SOL1 and SOL2 which is obtained when SOLUTION1 is mixed with MIXSOL2DISPPOL or SOL2.

SDD is a spray dried solid dispersion of AA in DISPPOL. AA and DISPPOL are preferably homogeneously mixed in SDD.

In a solid dispersion of AA in DISPPOL, AA may be homogeneously and preferably also molecularly dispersed in DISPPOL. AA and DISPPOL may form a solid solution in SDD.

AA is amorphous or substantially amorphous in SDD; substantially means that at least 80 wt %, preferably at least 90 wt %, more preferably at least 95 wt %, even more preferably at least 98 wt %, especially at least 99% wt %, of AA is amorphous; the wt % being based on the total weight of AA in SDD. SDD therefore may be an amorphous SDD. The amorphous nature of AA may be evidenced by a lack of sharp Bragg diffraction peaks in the x-ray pattern when SDD is analyzed by a powder X-Ray Diffraction (PXRD). Possible parameters and settings for a x-ray diffractometer are equipment with a Cu-Kalpha source, setting in modified parallel beam geometry between 3 and 40° 2Theta and a scan rate of 2°/min with a 0.0° step size. Another evidence for the amorphous nature of AA in the SDD may be a single glass transition temperature (Tg). A single Tg is also evidence of a homogeneous mixture of amorphous AA and polymer. Samples as such without any further sample preparation may be used for the determination of the Tg, the determination may run for example in modulated mode at a scan rate of 2.5°C./min, modulation of ±1.5°C./min, and a scan range from 0 to 180° C. Amorphous nature of AA shows a Tg which is equal to the Tg of neat DSISPPOL or which is between the Tg of the polymer and the Tg of the AA. The Tg of the SDD is often similar to the weighted average of the Tg of AA and the Tg of DISPPOL. SDD is amorphous or substantially, SDD can also be called ASD.
The concentration of DISPPOL in SUPSATSOL may be above or below, preferably below the saturation concentration of DISPPOL in SOLMIX at a given temperature, in particular at the temperature of SUPSATSOL when SUPSATSOL is fed into the spray dryer.
In one embodiment, DISPPOL is present in SUPSATSOL in a dissolved state, the amounts of DISPPOL and SOLMIX are chosen respectively.

Amounts of DISPPOL in SUPSATSOL may be from 0.5 wt % to 20 wt %, preferably from 1 wt % to 20 wt %, more preferably from 2.5 wt % to 15 wt %, even more preferably from 5 wt % to 10 wt %, with the wt % being based on the weight of SUPSATSOL.

The SDD may comprise from 1 to 99 wt %, preferably from 10 to 95 wt %, more preferably from 10 to 80 wt %, even more preferably from 20 to 60 wt %, of AA, the wt % being based on the weight of the SDD.

The SDD may comprise from 1 to 99 wt %, preferably from 20 to 90 wt %, more preferably 40 to 80 wt %, of DISPPOL, the wt % being based on the weight of the SDD.

Preferably, the combined content of AA and DISPPOL in SDD is from 65 to 100 wt %, more preferably from 67.5 to 100 wt %, even more preferably from 80 to 100 wt %; especially from 90 to 100 wt %; more especially from 95 to 100 wt %;
the wt % being based on the weight of the SDD;
in one embodiment, the SDD consists of AA and DISPPOL.

Relative amounts of AA to DISPPOL in SDD may be from 50:1 to 1:50, preferably from 25:1 to 1:25, more preferably from 10:1 to 1:10 (w/w).
AA may be any biologically active compound. The biologically active compound may be desired to be administered to a patient in need of the active agent.
AA may be a drug, medicament, pharmaceutical, therapeutic agent, nutraceutical or an active pharmaceutical ingredient (API).
AA may be a “small molecule,” generally having a molecular weight of 2000 Daltons or less.
AA may be a Bronstedt base with a basic pK_{A} of at least 5 or larger. Preferably, when AA is a Bronstedt base, then AA is combined with the acetic acid in its free base form. AA may be present in SUPSATSOL in its free base form or in its protonated form. When AA is combined with the acetic acid in its free base form, then AA is obtained again in its free base form after the spray drying.
AA may have a solubility of 40 mg/ml or less in SOL2 at a given temperature, in particular at the temperature of SUPSATSOL when SUPSATSOL is fed into the spray dryer.
AA may be nilotinib.
AA may be one or more active agents; SDD may contain one or more AA.
   DISPPOL may comprise one or more dispersion polymers, preferably 1, 2, 3 or 4, more preferably 1, 2 or 3, even more preferably 1 or 2 dispersion polymers.
   DISPPOL may be a pharmaceutically acceptable dispersion polymer.
   Suitable DISPPOL include, but are not limited to, hydroxypropyl methylcellulose acetate succinate (HPMCAS), hydroxypropyl methylcellulose phthalate (HPMCP), hydroxypropyl methyl cellulose (HPMC), hydroxypropyl cellulose (HPC), cellulose acetate phthalate (CAP), carboxymethyl ethyl cellulose (CMEC), polyvinylpyrrolidone (PVP), poly(vinylpyrrolidone-co-vinyl acetate) (PVP-VA), poly(methacrylic acid-co-methyl methacrylate) (PMMAMAA), poly(methacrylic acid-co-ethyl acrylate), or any combination thereof.
   Suitable PMMAMAA polymers include, but are not limited to, poly(methacrylic acid-co-methyl methacrylate) 1:1 (for example Eudragit® L100), and poly(methacrylic acid-co-methyl methacrylate) 1:2 (for example Eudragit® S100). Eudragit® are polymer products of Evonik Industries AG, 45128 Essen, Germany.
   The poly(methacrylic acid-co-ethyl acrylate) may be poly(methacrylic acid-co-ethyl acrylate) 1:1.

In some embodiments, DISPPOL is HPMCAS or PVP-VA.
SOL1 has only one liquid phase.
SOL1 may comprise from 90 to 100 wt %, preferably from 95 to 100 wt %, more preferably from 97.5 to 100 wt %, even more preferably from 98 to 100 wt %, especially from 99 to 100 wt %, of acetic acid, with the wt % being based in the weight of SOL1.
Preferably, SOL1 consists of acetic acid. SOL1 may be glacial acetic acid.
When SOL1comprises less than 100 wt % of acetic acid, then SOL1 may comprise besides acetic acid further solvents such as water, methanol, ethanol, 1-propanol, 2-propanol, acetone, 2-butanone, THF, methyl acetate, ethyl acetate, dichloromethane, 1,3-dioxolane, or mixtures thereof;
preferably water, methanol, acetone, or mixtures thereof;
more preferably water.
SOLUTION1 is prepared by dissolving AA in SOL1 and optionally adding any DISPPOL.
SOLUTION1 may be prepared by dissolving AA in SOL1, and optionally adding any DISPPOL, at a temperature from 4° C. to the boiling point of SOL1 at ambient pressure, preferably from 4° C. to a temperature below the boiling point of SOL1 at ambient pressure, more preferably at a temperature from room temperature to 60° C.
AA in SOLUTION1 is in a dissolved state in SOLUTION1, the amounts of AA and of SOL1 are chosen respectively.
The concentration of AA in SOLUTION1 is below the saturation concentration of AA in SOL1 at a given temperature, in particular at the temperature of SOLUTION1 when SOLUTION1 is mixed with MIXSOL2DISPPOL or with SOL2 respectively to provide SUPSATSOL.
In one embodiment, DISPPOL is present in SOLUTION1 in a dissolved state, the amount of DISPPOL is chosen respectively.
The concentration of DISPPOL in SOLUTION1 is preferably below the saturation concentration of DISPPOL in SOL1 at a given temperature, in particular at the temperature of SOLUTION1 when SOLUTION1 is mixed with MIXSOL2DISPPOL or with SOL2 respectively to provide SUPSATSOL.

Typical solubility of AA in SOL1 may be at least 1 wt %, preferably at least 2 wt %, more preferably at least 5 wt %, even more preferably at least 10 wt %, especially at least 20 wt %, of AA; with the wt % being based on the weight of SOLUTION1; the solubility of AA in SOL1 being preferably at a temperature of from 4° C. to the boiling point of SOL1 at ambient pressure, preferably from 4° C. to a temperature below the boiling point of SOL1 at ambient pressure, more preferably from room temperature to 60° C. SOL1may be chosen respectively.

Lower limit of the amount of AA in SOLUTION1 may be at least 0.5 wt %, preferably at least 1 wt %, more preferably at least 2.5 wt %, even more preferably at least 5 wt %, especially at least 7.5 wt %, more especially at least 10 wt %, even more especially at least 20 wt %, in particular at least 30 wt %, with the wt % being based on the weight of SOLUTION1.

The amount of AA in SOLUTION1 may be up to 50 wt %, preferably up to 40 wt %, even more preferably up to 35 wt %, with the wt % being based on the weight of SOLUTION1.

Any of the lower limit of the amount of AA in SOLUTION1 may be combined with any of the upper limit of the amount of AA in SOLUTION1.

For example, amounts of AA in SOLUTION1 may be from 0.5 to 50 wt %, preferably from 0.5 to 40 wt %, more preferably from 0.5 to 35 wt %, with the wt % being based on the weight of SOLUTION1.
SOL2 has only one liquid phase.
SOL2 is a solvent commonly used for spray drying.
SOL2 may comprise methanol, ethanol, 1-propanol, 2-propanol, acetone, 2-butanone, THF, methyl acetate, ethyl acetate, dichloromethane, 1,3-dioxolane, or mixtures thereof.
SOL2 may comprise water in such an amount that SOL2 remains having only one liquid phase. The solubilities of water in the possible non-aqueous solvents of SOL2 are known. Depending on the possible non-aqueous solvent SOL2, SOL2 may comprise 25 wt % or less of water, the wt % being based in the weight of SOL2.
Preferably SOL2 comprises methanol, ethanol, acetone, or mixtures thereof, preferably with SOL2 comprising from 0 to 25 wt % water, the wt % being based in the weight of SOL2.
More preferably SOL2 is methanol, acetone or mixtures thereof, preferably with SOL2 comprising from 0 to 25 wt % water, the wt % being based in the weight of SOL2.
Even more preferably SOL2 is methanol, preferably with SOL2 comprising from 0 to 25 wt % water, the wt % being based in the weight of SOL2.
MIXSOL2DISPPOL is prepared by mixing DISPPOL with SOL2.
MIXSOL2DISPPOL may be prepared by mixing DISPPOL with SOL2 at a temperature from 4° C. to the boiling point of SOL2 at ambient pressure, preferably from 4° C. to a temperature below the boiling point of SOL2 at ambient pressure, more preferably at a temperature from room temperature to 60° C.
In one embodiment, DISPPOL is present in MIXSOL2DISPPOL in a dissolved state, the amounts of DISPPOL is chosen respectively.
The concentration of DISPPOL in MIXSOL2DISPPOL is preferably below the saturation concentration of DISPPOL in SOL2 respectively at a given temperature, in particular at the temperature of MIXSOL2DISPPOL when MIXSOL2DISPPOL is mixed with SOLUTION1 to provide SUPSATSOL.

SOL2 may have a boiling point at ambient pressure of 115° C. or less.

Amounts of DISPPOL in MIXSOL2DISPPOL or in SOLUTION1 may be from 0.5 wt % to 20 wt %, preferably from 1 wt % to 20 wt %, more preferably from 2.5 wt % to 15 wt %, even more preferably from 5 wt % to 10 wt %, with the wt % being based on the weight of MIXSOL2DISPPOL or of SOLUTION1 respectively.

AA may have a solubility in SOL1 that is at least 5-fold, preferably at least 10-fold, more preferably at least 50-fold, even more preferably at least 100-fold higher than the solubility of AA in SOL2 at a given temperature, in particular at the temperature of SOLUTION1 when SOLUTION1 is mixed with MIXSOL2DISPPOL or with SOL2 to provide SUPSATSOL; SOL1 and SOL2 may be chosen respectively.
The ratio (w:w) of the amounts of SOL1:SOL2, when SUPSATSOL is prepared by mixing SOLUTION1 with MIXSOL2DISPPOL or with SOL2, may be from 1:1 to 1:20, preferably from 1:2 to 1:20, more preferably from 1:5 to 1:20, even more preferably from 1:8 to 1:20, especially from 1:8 to 1:15, more especially from 1:8 to 1:10.
The lower limit of the amount of SOL1 in SOLMIX may be 5 wt %, preferably 6 wt %, more preferably 7.5 wt %, the wt % being based on the weight of SOLMIX.
The upper limit of the amount of SOL1 in SOLMIX may be 50 wt %, preferably 33 wt %, more preferably 25 wt %, even more preferably 20 wt %, especially 15 wt %, more especially 10 wt %, the wt % being based on the weight of SOLMIX.
For ranges of the amount of SOL1 in SOLMIX any of the lower limits may be combined with any of the upper limits, for example the amount of SOL1 in SOLMIX may be from 5 to 50 wt %, preferably from 5 to 33 wt %, more preferably from 5 to 25 wt %, even more preferably from 5 to 20 wt %, especially from 5 to 15 wt %, more especially from 5 to 10 wt %, the wt % being based on the weight of SOLMIX.
SUPSATSOL may be fed into the spray dryer with a temperature of SUPSATSOL up to the boiling point of SUPSATSOL at ambient pressure; preferably with a temperature of from 4° C. to the boiling point of SUPSATSOL at ambient pressure, preferably from 4° C. to a temperature below the boiling point of SUPSATSOL at ambient pressure, more preferably from room temperature to 60° C. In the context of this invention the term “SUPSATSOL may be fed into the spray dryer with a temperature of SUPSATSOL” means that “SUPSATSOL is spray dried with a temperature of SUPSATSOL”.

The spray drying of SUPSATSOL in the spray dryer evaporates both SOL1 and SOL2.

The temperatures
for preparing SOLUTION1,
for preparing MIXSOL2DISPPOL,
for preparing SUPSATSOL, and
for feeding SUPSATSOL into the spray dryer may be same of different, they may be from room temperature to 60° C.; preferably, the temperature
for preparing SOLUTION1 may be from room temperature to 60° C.,
for preparing MIXSOL2DISPPOL may be room temperature,
for preparing SUPSATSOL may be from room temperature to 60° C., and
for feeding SUPSATSOL into the spray dryer may be from room temperature to 60° C.

The spray drying may be done with an inlet temperature of from 80 to 165° C.
The spray drying may be done with an outlet temperature equal to or less than the boiling point of the solvent in in SOLMIX that has the highest boiling point.
The spray drying may be done with any inert gas commonly used for spray drying, such as nitrogen.

SUPSATSOL may further comprises a surfactant SURF.

SURF may be mixed with SUPSATSOL, or SURF may be mixed with SOLUTION1, with MIXSOL2DISPPOL, with SOL1 or with SOL2 before the preparation of SUPSATSOL. SURF may be for example a fatty acid and alkyl sulfonate; docusate sodium (available from Mallinckrodt Spec. Chern., St. Louis, Mo.), and polyoxyethylene sorbitan fatty acid esters (Tween®, available from ICI Americas Inc, Wilmington, Del., Liposorb® P-20, available from Lipochem Inc, Patterson, N.J., and Capmul® POE-0, available from Abitec Corp., Janesville, Wis.), and natural surfactants such as sodium taurocholic acid, 1-palmitoyl-2-oleoyl-sn-glycero-3-phosphocholine, lecithin, other phospholipids and mono- and diglycerides, vitamin E TPGS, PEO, PEO-PPO-PEO triblock copolymers (known under the tradename pluronics), and PEO (PEO are also called PEG, polyethyleneglycols (PEG)).

The amount of SURF may be up to 10 wt %, the wt % being based on the weight of SDD.

SUPSATSOL may further comprises pharmaceutically acceptable excipients, such as fillers, disintegrating agents, pigments, binders, lubricants, flavorants, and so forth which can be used for customary purposes and in typical amounts known to the person skilled on the art.

The viscosity of SUPSATSOL may be at least 2 times, preferably at least 3 times, lower than the viscosity of a mixture of DISPPOL in SOL1 which has the same concentration of DISPPOL as the concentration of DISPPOL is in SUPSATSOL.

The viscosity of SUPSATSOL may be at least 2 times, preferably at least 3 times, lower than the viscosity of SOLUTION1.

After the spray drying of SUPSATSOL the SDD may be submitted to a second drying in order to reduce the amount of any residual SOL1 or SOL2 in SDD. Secondary drying may be done using a tray dryer or any agitated dryer known to the skilled person for drying solids.
Preferably, the final SDD may have a content of SOL1 of 5000 ppm or less, preferably of 500 ppm or less, more preferably of 100 ppm or less.
Preferably, the final SDD may have a content of SOL2 of 5000 ppm or less, preferably of 500 ppm or less, more preferably of 100 ppm or less.
Further subject of the invention is a spray dried solid dispersion SDD; wherein the SDD is obtainable by the method SPRAYDRY;
with SDD and SPRAYDRY as defined herein, also with all their embodiments.

### EXAMPLES

### Materials and Abbreviations

cP centipoise, centipoise is equal to the SI millipascal seconds (mPa·s)
HPMCAS-MG HPMCAS in form of AQOAT® MG (also called AS-MG) was purchased from Shin-Etsu Chemical Co., Ltd. (Tokyo, Japan). The letter M specifies the grade and distinguish the contents of acetyl and succinoyl groups. Other grades are designated with the letters L (HPMCAS-L) and H (HPMCAS-H). The Letter G represents granular grade with a Mean Particle Size of 1 mm, a letter F instead of a G would represent micronized grade with a Mean Particle Size of 5 micrometer. Various contents and parameters of these grades are given in Table 3.

TABLE 3

| | | | Hydroxy- | Acetyl | Succinoyl | |
|---|---|---|---|---|---|---|
| | | Methoxy | propoxy | content [wt %] | content [wt %] | |
| | Viscosity | content | content | range/ | range/ | Tg |
| Grade | (mPa*s) (a) | [wt %] (c) | [wt %] (c) | preferred (c) | preferred (c) | [° C.] (b) |
| L | 2.4 to 3.6 | 20 to 24 | 5 to 9 | 5 to 9/6 | 14 to 18/15 | 122 |
| M | 2.4 to 3.6 | 21 to 25 | 5 to 9 | 7 to 11/8 | 10 to 14/11 | 122 |
| H | 2.4 to 3.6 | 22 to 26 | 6 to 10 | 10 to 14/12 | 4 to 8/6 | 122 |

(a) Viscosity of 2 w/w % solution of sodium hydroxide aqueous solution at 20° C.
(b) Tg of the HPMCAS was determined by DSC experiment under the following test condition:
Equipment: DSC Q2000 (TA Instruments. Japan)
Heating rate: 10° C./min
Referred to the second heating run
N₂ gas atmosphere
Sample size 3 mg
(c) the wt % based on the weight of the HPMCAS

Nilotinib CAS 641571-10-0; Nilotinib, Free Base, >99%, was purchased from LC Laboratories, Woburn, MA 01801, USA. The term “nilotinib” refers to the free base form throughout the examples, if not explicitly stated otherwise. Nilotinib has two basic pKA: a basic pK_{A} of 2.1 and a basic pK_{A} of 5.4. At each of these basic pKA half of the respective basic site is protonated.

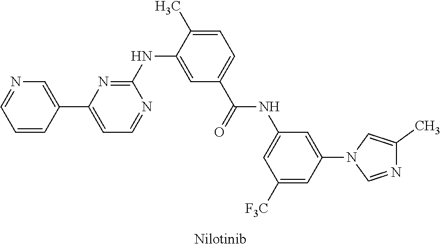 [SMILES: Cc1cn(-c2cc(NC(=O)c3ccc(C)c(Nc4nccc(-c5cccnc5)n4)c3)cc(C(F)(F)F)c2)cn1]

PVP-VA64 Kollidon® VA64, Vinylpyrrolidone-vinyl acetate copolymer, CAS 25086-89-9, PVP/VA Copolymer, BASF, Ludwigshafen, Germany

### METHODS

### Solubility Determination of Nilotinib

A saturated solution of nilotinib was prepared in the respective solvent at the respective temperature with excess crystalline nilotinib and allowed to stir for 24 h, a suspension of nilotinib in the solvent, which was saturated with nilotinib, was obtained. The suspension was filtered through a 1 micrometer glass filter. Crystalline solubility was determined by analysis of the filtrate, which is the saturated solution, by gravimetry (weighing).

Details and results are given in Table 1.

TABLE 1

| | Solubility at | Solubility at |
|---|---|---|
| Solvent | 25° C. (1) | 40° C. (1) |
| Methanol | 2.8 mg/ml - - - 0.36 wt % | 5.0 mg/ml - - - 0.66 wt % |
| Methanol:Acetic | 6 mg/ml - - - 0.76 wt % | 8 mg/ml - - - 1.0 wt % |
| Acid 9:1 (w:w) | | |
| Glacial | 135 mg/ml - - - 12.7 wt % | 233 mg/ml - - - 21.1 wt % |
| Acetic Acid | | |

(1) Solubilities are given in
mg of nilotinib per ml saturated solution
wt % of nilotinib based on the weight of the saturated solution
The wt % and mg/mL values are not precisely in alignment, since they were measured separately. In case of doubt the wt % value prevails.

### Viscosity of Solutions of Polymers in Solvents

The viscosities of solutions of polymer in solvents were measured by adding 400 g of solvent to a 500 mL jacketed vessel set to 20° C., stirring the polymer into the solvent to several weight percentages between 0 and 20 wt %, and measuring viscosity at each weight percent with a Hydramotion ReactaVisc viscometer, Hydramotion Ltd., York, UK. The viscosity at 9 wt % polymer, based on the weight of the solutions of polymer in solvents, was interpolated.

Table 2 shows interpolated viscosity [cP] of polymers at 9 wt % in respective solvents.

TABLE 2

| | | Viscosity [cP] | |
|---|---|---|---|
| | | MeOH:AcOH | Glacial |
| Polymer | MeOH | 9:1 (w:w) | AcOH |
| HPMCAS | 14.6 | 16.5 | 65.5 |
| PVP-VA64 | 1.8 | 1.9 | 9.0 |

### Example 1—Nilotinib Concentration Enhancement in Methanol:Acetic Acid Using Solvent-Shift

The Solvent-Shift route was done as follows:

A 19.9 wt % solution of nilotinib in glacial acetic acid was prepared at 40° C. by dissolving 0.521 g nilotinib in 2.10 g acetic acid. 0.218 g of this solution was added to 1.80 g of methanol at 40° C., resulting in a supersaturated solution of 2.15 wt % nilotinib in 91.2:8.8 (w:w) methanol:acetic acid. This final supersaturated solution of nilotinib in SOLMIX using this Solvent Shift procedure remained visibly clear without any precipitation of solid for at least 4 h. Visible solids were observed at 7.5 h.

As a control a 2.06 wt % mixture of nilotinib in 89.5:10.5 (w:w) methanol:acetic acid was also prepared; weighing the nilotinib directly into 89.5:10.5 (w:w) methanol:acetic acid at 40° C. The control mixture resulted in a visible slurry, that is a suspension of undissolved nilotinib, whereas the final supersaturated solution prepared via the Solvent Shift route was visibly clear without any undissolved solids for up to 4 h.
SOL1: glacial acetic acid
SOL2: methanol
SOLMIX: mixture of acetic acid and methanol with ca. 9 wt % acetic acid based on the weight of SOLMIX.

### Example 2—Nilotinib Concentration Enhancement in Methanol:Acetic Acid in the Presence of HPMCAS-MG Polymer Using Solvent-Shift

The Solvent-Shift route was done as follows:

A 19.9 wt % solution of nilotinib in glacial acetic acid was prepared at 40° C. by dissolving 0.521 g nilotinib in 2.10 g acetic acid.

A 6.77 wt % solution of HPMCAS-MG in methanol was prepared at room temperature by dissolving 0.643 g HPMCAS-MG in 8.86 g methanol. This solution and was then heated to 40° C.

0.217 g of the solution of nilotinib in acetic acid at 40° C. was added to 1.81 g of the solution of HPMCAS-MG in methanol at 40° C., resulting in a final supersaturated solution of 2.13 wt % nilotinib in 85.0:8.8:6.2 (w:w:w) methanol:acetic acid:HPMCAS-MG at 40° C. The final supersaturated solution of nilotinib in SOLMIX remained visibly clear without any precipitation of solid for at least 22 h.
SOL1: glacial acetic acid
SOL2: methanol
SOLMIX: mixture of acetic acid and methanol with ca. 9 wt % acetic acid based on the weight of SOLMIX.

### Example 3—Nilotinib Concentration Enhancement in Methanol:Acetic Acid in the Presence of PVP-VA64 Polymer using Solvent-Shift

The Solvent-Shift route was done as follows:

A 19.9 wt % solution of nilotinib in glacial acetic acid was prepared at 40 ° C. by dissolving 0.521 g nilotinib in 2.10 g acetic acid. A 6.58 wt % solution of PVP-VA64 in methanol was prepared at room temperature by dissolving 0.651 g polymer in 9.24 g methanol and was then heated to 40° C.

0.223 g of the nilotinib solution in acetic acid at 40° C. was added to 1.80 g of the PVP-VA64 solution in methanol at 40° C., resulting in a final supersaturated solution of 2.19 wt % nilotinib in 85.0:9.0:6.0 (w:w:w) methanol:acetic acid:PVP-VA64 at 40° C. The final supersaturated solution of nilotinib in SOLMIX remained visibly clear without any precipitation of solid for at least 2 h. Visible solids were observed at 4 h.
SOL1: glacial acetic acid
SOL2: methanol
SOLMIX: mixture of acetic acid and methanol with ca. 10 wt % acetic acid based on the weight of SOLMIX.

### Example 4—Nilotinib/HPMCAS SDD Using Solvent-Shift

The Solvent-Shift route was done as follows:

7.5 g of HPMCAS-MG were mixed in 90 g of methanol at room temperature resulting in a 7.7 wt % solution.

2.5 g of nilotinib were dissolved at 50° C. in 10 g of glacial acetic acid resulting in a 20 wt % solution.

The solution of nilotinib in glacial acetic acid with a temperature of 50° C. was added to the mixture of HPMCAS-MG in methanol under stirring at room temperature over the course of 2 min providing a final supersaturated solution SUPSATSOL of nilotinib in SOLMIX which was in 9:1 (w:w) methanol:acetic acid. The final supersaturated solution had a nilotinib concentration of 2.27 wt % and a HPMCAS-MG concentration of 6.82 wt %. The supersaturated solution continued to stir for approximately 10 min before it was sprayed, it did not contain nilotinib in solid form, instead it contained the nilotinib in a completely dissolved state, and it had only one liquid phase.

For the spraying the supersaturated solution having room temperature was pumped at room temperature using a peristaltic pump into a lab-scale 0.3 m diameter stainless steel spray drying chamber. The flow rate of the supersaturated solution was 20 g/min., atomization was done through a two-fluid nozzle ¼ J series with an 1650 air cap and a 54 liquid cap made by Spraying Systems Company, Glendale Heights, IL 60187-7901, United States. Heated nitrogen gas was introduced into the 0.3 m diameter stainless steel spray drying chamber at a temperature of 130° C. and flow rate of 500 g/min. The outlet temperature of the gas exiting the chamber was 45 to 48° C. The spray drying provided a SDD which was collected using a cyclone to separate the solid particles from the gas stream.
SOL1: glacial acetic acid
SOL2: methanol
SOLMIX: mixture of acetic acid and methanol with 10 wt % acetic acid based on the weight of SOLMIX.

The SDD was amorphous.

1. A method for preparing a spray dried solid dispersion comprising an active agent and a dispersion polymer, the method comprising:
providing a first solution of the active agent in a first solvent;
mixing the first solution with a second solvent to provide a supersaturated solution; and
spray drying the supersaturated solution using a spray dryer,
wherein
the active agent is a drug, medicament, pharmaceutical, therapeutic agent, nutraceutical or an active pharmaceutical ingredient,
the supersaturated solution comprises a solvent mixture and the active agent, with the solvent mixture being a mixture of the first solvent and the second solvent,
the supersaturated solution is a supersaturated solution of the active agent in solvent mixture,
the supersaturated solution does not contain the active agent in solid form,
the dispersion polymer is contained in the first solution, in the second solvent or in both prior to the mixing of the first solution with the second solvent,
the first solvent comprises from 90 wt % to 100 wt % of acetic acid, with the wt % being based on a weight of the first solvent, and
the active agent is stable in the first solvent, the second solvent and the solvent mixture.

2. The method according to claim 1, wherein the dispersion polymer is a pharmaceutically acceptable dispersion polymer.

3. The method according to claim 1, wherein the dispersion polymer includes hydroxypropyl methylcellulose acetate succinate (HPMCAS), hydroxypropyl methylcellulose phthalate, hydroxypropyl methyl cellulose, hydroxypropyl cellulose, cellulose acetate phthalate, carboxymethyl ethyl cellulose, polyvinylpyrrolidone, poly(vinylpyrrolidone-co-vinyl acetate) (PVP-VA), poly(methacrylic acid-co-methyl methacrylate), poly(methacrylic acid-co-ethyl acrylate), or any combination thereof.

4. The method according to claim 1, wherein the dispersion polymer is HPMCAS or PVP-VA.

5. The method according to claim 1, wherein the first solvent consists of acetic acid.

6. The method according to claim 1, wherein the second solvent comprises methanol, ethanol, 1-propanol, 2-propanol, acetone, 2-butanone, tetrahydrofuran (THF), methyl acetate, ethyl acetate, dichloromethane, 1,3-dioxolane, or any mixture thereof.

7. The method according to claim 1, wherein the second solvent comprises methanol, ethanol, acetone, or any mixture thereof.

8. The method according to claim 1, wherein a ratio (w:w) of amounts of the first solvent:the second solvent, when the supersaturated solution is prepared by mixing first solution with a mixture of the second solvent and the dispersion polymer or with the second solvent, is from 1:1 to 1:20.

9. A spray dried solid dispersion obtained by the method of claim 1.

10. The method according to claim 1, wherein:
the first solvent consists of acetic acid; and
the second solvent comprises methanol, ethanol, 1-propanol, 2-propanol, acetone, 2-butanone, THF, methyl acetate, ethyl acetate, dichloromethane, 1,3-dioxolane, or any mixture thereof.

11. The method according to claim 10, wherein the second solvent comprises methanol, ethanol, acetone, or any mixture thereof.

12. The method according to claim 10, wherein the dispersion polymer comprises includes HPMCAS, hydroxypropyl methylcellulose phthalate, hydroxypropyl methyl cellulose, hydroxypropyl cellulose, cellulose acetate phthalate, carboxymethyl ethyl cellulose, PVP-VA, poly(methacrylic acid-co-methyl methacrylate), poly(methacrylic acid-co-ethyl acrylate), or any combination thereof.

13. The method according to claim 10, wherein the dispersion polymer is HPMCAS or PVP-VA.

14. The method according to claim 10, wherein a ratio (w:w) of amounts of the first solvent:the second solvent is from 1:1 to 1:20, when the supersaturated solution is prepared by mixing first solution with a mixture of the second solvent and the dispersion polymer or with the second solvent.

15. A spray dried solid dispersion obtained by the method of claim 10.

16. The method according to claim 3, wherein the first solvent consists of acetic acid.

17. The method according to claim 3, wherein the second solvent comprises methanol, ethanol, 1-propanol, 2-propanol, acetone, 2-butanone, tetrahydrofuran (THF), methyl acetate, ethyl acetate, dichloromethane, 1,3-dioxolane, or any mixture thereof.

18. The method according to claim 3, wherein the second solvent comprises methanol, ethanol, acetone, or any mixture thereof.

19. The method according to claim 3, wherein a ratio (w:w) of amounts of the first solvent:the second solvent is from 1:1 to 1:20, when the supersaturated solution is prepared by mixing first solution with a mixture of the second solvent and the dispersion polymer or with the second solvent.

20. A spray dried solid dispersion obtained by the method of claim 3.
